(19) United States
(12) Patent Application Publication
(10) Pub. No.: US 2023/0001144 A1
(43) Pub. Date: Jan. 5, 2023

(54) Catheter Assembly Adapter, Instrument Delivery Device, and Related Methods

(71) Applicant: Becton, Dickinson and Company, Franklin Lakes, NJ (US)

(72) Inventors: Jonathan Karl Burkholz, Salt Lake City, UT (US); John Lackey, West Valley City, UT (US); Erica E. Neumann, Salt Lake City, UT (US); Weston F. Harding, Lehi, UT (US)

(21) Appl. No.: 17/851,689

(22) Filed: Jun. 28, 2022

Related U.S. Application Data
(60) Provisional application No. 63/218,127, filed on Jul. 2, 2021.

(51) Int. Cl. A61M 25/00; A61M 25/06

(52) U.S. Cl. CPC A61M 25/0097; A61M 25/0606; A61M 25/0014

(57) ABSTRACT

A catheter assembly may include an adapter having a distal end, a proximal end, a side port, and an adapter lumen. The catheter assembly may include a catheter hub, which may include a distal end, a proximal end, a catheter hub lumen, and another side port. The catheter assembly may include an extension tube. A distal end of the extension tube may be coupled to the other side port, and the distal end of the adapter may be coupled to a proximal end of the extension tube. A fluidic seal may be formed around a tube of an instrument advancement device when the tube is advanced into the adapter. As an example, a portion of the adapter lumen between the distal end of the adapter and the side port may include an annular protrusion configured to form the fluidic seal.

### CROSS-REFERENCE TO RELATED APPLICATION

The present application claims priority to U.S. Provisional Application No. 63/218,127, entitled “Catheter Assembly Adapter, Instrument Delivery Device, and Related Methods”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. More particularly, in some embodiments, the present disclosure relates to an adapter of a catheter assembly, an instrument delivery device, and related methods. In some embodiments, a catheter assembly may include the adapter, which may include a distal end, a proximal end aligned with the distal end of the adapter, a side port disposed between the distal end of the adapter and the proximal end of the adapter, and an adapter lumen. In some embodiments, the adapter lumen may extend through the distal end of the adapter, proximal end of the adapter, and the side port.

In some embodiments, the catheter assembly may include a catheter hub, which may include a distal end, a proximal end, a catheter hub lumen extending through the distal end of the catheter hub and the proximal end of the catheter hub, and another side port disposed in fluid communication with the catheter hub lumen.

In some embodiments, the catheter assembly may include an extension tube, which may include a distal end and a proximal end. In some embodiments, the distal end of the extension tube may be coupled to the other side port. In some embodiments, the distal end of the adapter may be coupled to the proximal end of the extension tube. In some embodiments, a portion of the adapter lumen between the distal end of the adapter and the side port may include an annular protrusion configured to form a fluidic seal around a tube of an instrument advancement device.

In some embodiments, the adapter may include a T-connector or a Y-connector. In some embodiments, the catheter assembly may include another extension tube. In some embodiments, the side port is coupled to the other extension tube. In some embodiments, the portion of the adapter lumen between the distal end of the adapter and the side port may include a cylindrical uniform diameter portion proximate the proximal end of the extension tube and proximate an outward taper. In some embodiments, the annular protrusion may be proximal to the outward taper.

In some embodiments, the annular protrusion may be proximate the proximal end of the extension tube. In some embodiments, an edge of the protrusion proximate the proximal end of the extension tube may be disposed an equal or greater distance from a longitudinal axis of the adapter than an inner surface of the extension tube proximate the annular protrusion.

In some embodiments, the catheter assembly may include an annular elastomeric seal disposed within the adapter lumen between the proximal end of the extension tube and the side port. In some embodiments, the annular elastomeric seal may be configured to form a fluidic seal around a tube of an instrument advancement device. In some embodiments, an inner surface of the adapter forming the adapter lumen may include an annular groove, and the annular elastomeric seal is seated within the annular groove. In some embodiments, the annular elastomeric seal may include an O-ring. In some embodiments, the portion of the adapter lumen between the distal end of the adapter and the side port may include the cylindrical uniform diameter portion proximate the outward taper, and the annular elastomeric seal may be proximal to the outward taper.

In some embodiments, an inner surface of the extension tube may include an annular protrusion configured to form a fluidic seal around a tube of an instrument advancement device. In some embodiments, the inner surface of the extension tube may include one or more other annular protrusions.

In some embodiments, the instrument delivery device may include a housing, which may include a proximal end, a distal end, a lumen disposed between the proximal end and a distal end, and a slot disposed between the proximal end and the distal end. In some embodiments, the instrument delivery device may include an advancement element extending through the slot and configured to move linearly along the slot between a retracted position and an advanced position.

In some embodiments, the instrument delivery device may include an instrument such as a tube, which may include a first end and a second end. In some embodiments, when the advancement element is moved linearly along the slot from the retracted position to the advanced position, the second end of the instrument may be advanced beyond the distal end of the housing. In some embodiments, the distal end of the tube may include an annular protrusion or an outward flare. In some embodiments, the annular protrusion of the tube or the outward flare may be configured to form a fluidic seal within the adapter of the catheter assembly. In some embodiments, the tube may include an increased diameter portion configured to form a fluidic seal within an adapter of a catheter assembly.

In some embodiments, a method of blood collection may include advancing the tube distally within the adapter of the catheter assembly. In some embodiments, the adapter may include the side port. In some embodiments, in response to advancing the tube distally within the adapter of the catheter assembly, a fluidic seal may be formed around the tube distal to the side port within the adapter. In some embodiments, a distal end of the adapter may include an opening, and the fluidic seal may be formed proximal to the opening. In some embodiments, the tube may include the annular protrusion, and in response to advancing the tube distally within the adapter of the catheter assembly, the fluidic seal may be formed at the annular protrusion of the tube.

In some embodiments, the adapter may include the distal end, the proximal end aligned with the distal end of the adapter, the side port disposed between the distal end of the adapter and the proximal end of the adapter, and the adapter lumen. In some embodiments, the adapter lumen may extend through the distal end of the adapter, the proximal end of the adapter, and the side port. In some embodiments, the portion of the adapter lumen between the distal end of the adapter and the side port may include the annular protrusion, which may be configured to form the fluidic seal in response to advancing the tube distally within the adapter of the catheter assembly.

In some embodiments, an elastomeric O-ring is disposed within adapter lumen between the proximal end of the extension tube and the side port, and the elastomeric O-ring may form the fluidic seal around the tube of the instrument delivery device in response to advancing the tube distally within the adapter of the catheter assembly. In some embodiments, the annular protrusion of the extension tube may form the fluidic seal around the tube of the instrument delivery device in response to advancing the tube distally within the adapter of the catheter assembly.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example instrument delivery device, illustrating an example advancement element in an example initial or retracted position, according to some embodiments;

FIG. 1B is a cross-sectional view of the instrument delivery device of FIG. 1A;

FIG. 1C is an enlarged cross-sectional view of a portion of the instrument delivery device of FIG. 1A, according to some embodiments;

FIG. 1D is a cross-sectional view of the instrument delivery device of FIG. 1A along the line 1D-1D of FIG. 1A, according to some embodiments;

FIG. 1E is an enlarged view of a portion of FIG. 1D, according to some embodiments;

FIG. 1F is a cross-sectional view of an example catheter system that includes the instrument delivery device with the advancement element in the initial or retracted position, according to some embodiments;

FIG. 2A is a cross-sectional view of an example adapter, illustrating an example annular protrusion, according to some embodiments;

FIG. 2B is a cross-sectional view of the catheter system that includes the adapter and the annular protrusion, illustrating an example tube in an advanced position, according to some embodiments;

FIG. 3A is a cross-sectional view of the catheter system that includes the adapter and another example annular protrusion, illustrating the tube in the advanced position, according to some embodiments;

FIG. 3B is a cross-sectional view of the adapter, illustrating the other annular protrusion, according to some embodiments;

FIG. 3C is an enlarged portion of the cross-sectional view of FIG. 3A, according to some embodiments;

FIG. 4A is a cross-sectional view of the catheter system that includes the adapter and example annular elastomeric seals, according to some embodiments;

FIG. 4B is an enlarged portion of the cross-sectional view of FIG. 4A, according to some embodiments;

FIG. 5A is a cross-sectional view of the catheter system that includes the adapter and an example extension tube having one or more annular protrusions, illustrating the tube in the advanced position, according to some embodiments;

FIG. 5B is a cross-sectional view of the adapter, illustrating the extension tube with an example annular protrusion, according to some embodiments;

FIG. 6A is a cross-sectional view of the catheter system that includes the adapter and the tube having an example annular protrusion, illustrating the tube in the advanced position, according to some embodiments;

FIG. 6B is an upper perspective view of a distal end of the tube having the annular protrusion, according to some embodiments;

FIG. 6C is a cross-sectional view of the distal end of the tube having the annular protrusion, according to some embodiments;

FIG. 6D is a cross-sectional view of a portion of the catheter system, illustrating the tube having an enlarged diameter portion, according to some embodiments;

FIG. 7A is a cross-sectional view of the catheter system, illustrating the tube having an outward flare and disposed in the advanced position, according to some embodiments;

FIG. 7B is a cross-sectional view of the tube having the outward flare, according to some embodiments;

FIG. 8 is a cross-sectional view of an example catheter adapter and the tube having a distal end configured to seal a fluid pathway through the catheter adapter, according to some embodiments;

FIG. 9A is a cross-sectional view of the adapter, according to some embodiments;

FIG. 9B is a cross-sectional view of the adapter, according to some embodiments;

FIG. 9C is a cross-sectional view of the adapter, according to some embodiments;

FIG. 10A is a cross-sectional view of an example needle-free connector, according to some embodiments; and

FIG. 10B is a cross-sectional view of the needle-free connector coupled to an example connector, according to some embodiments, according to some embodiments.

### DETAILED DESCRIPTION

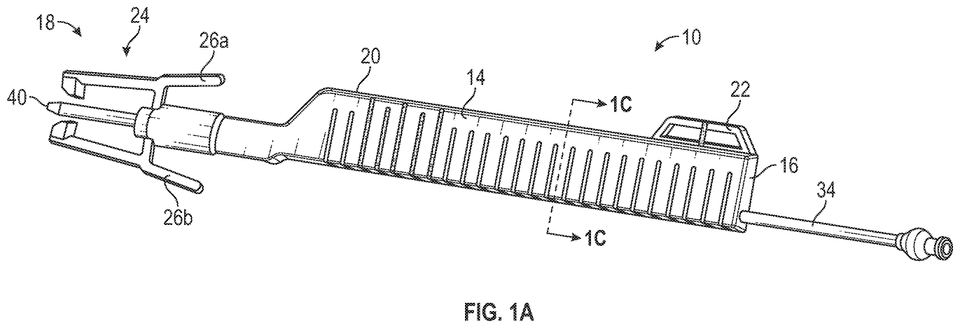

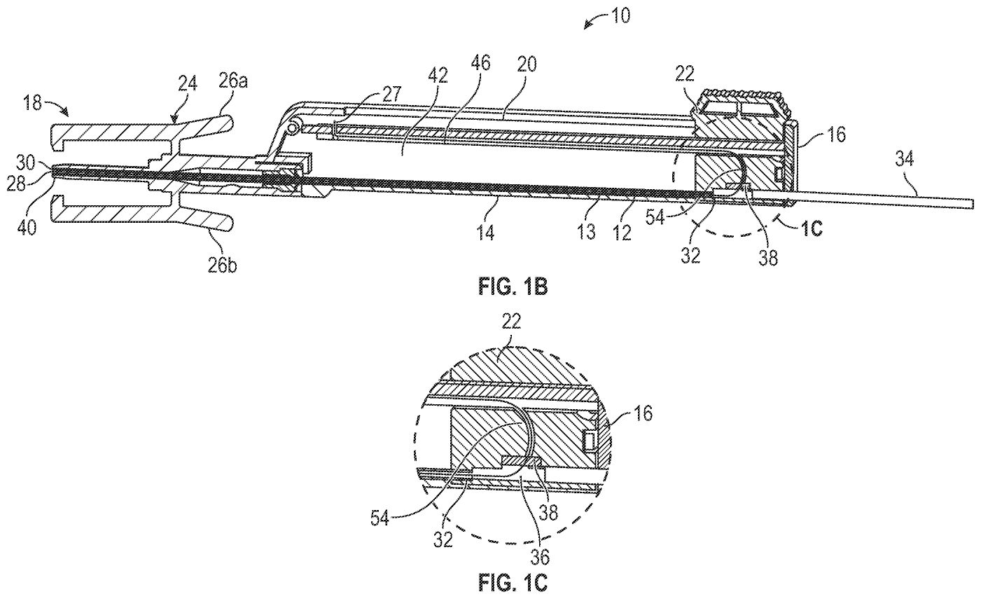

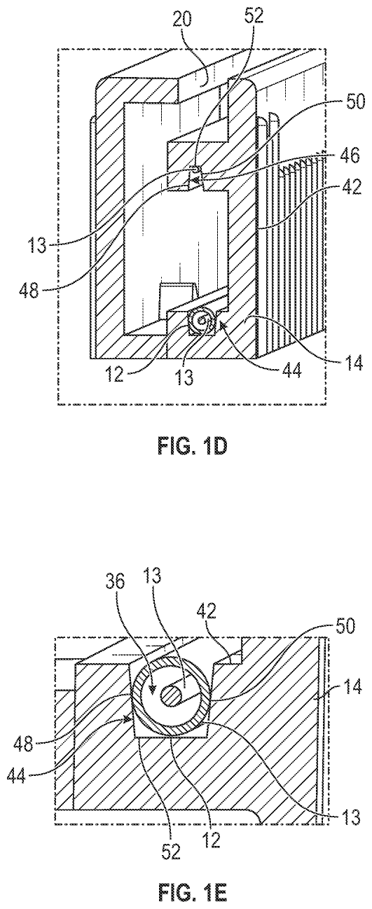

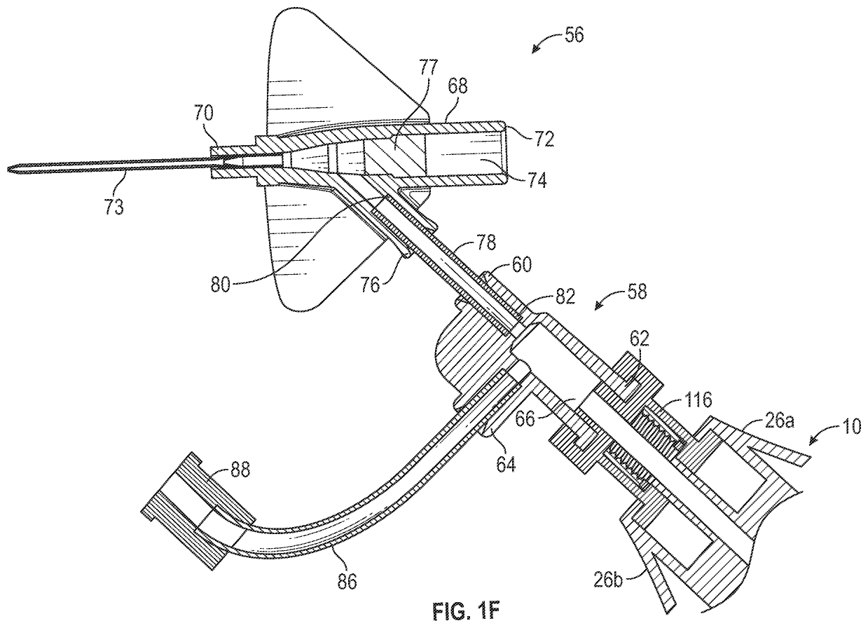

Referring now to FIGS. 1A-1E, in some embodiments, an instrument delivery device 10 may be configured to deliver a tube 12 and/or a guidewire 13 into or through a catheter assembly. In some embodiments, the instrument delivery device 10 may provide needle-free delivery of the tube 12 and/or the guidewire 13 into or through the catheter assembly. In some embodiments, the tube 12 and/or the guidewire 13 may be advanced through a catheter of the catheter assembly to push past any occlusions in the catheter or vasculature (e.g., a thrombus or a fibrin sheath at a tip of the catheter, vein collapse, valves, etc.) to create a clear pathway for fluid flow into the catheter assembly, which may aid in blood collection. In some embodiments, the tube 12 and/or a guidewire 13 may reduce or remove occlusions, improving patency of the catheter for medication and fluid delivery, as well as blood acquisition during a dwell time of the catheter. In some embodiments, the instrument delivery device 10 may improve a blood collection flow rate, blood sample quality, and fluid path robustness, while maintaining a small size to facilitate handling by a user.

In some embodiments, the catheter may include a peripheral intravenous (IV) catheter, a peripherally-inserted central catheter, or a midline catheter. In some embodiments, the catheter through which the tube 12 and/or the guidewire 13 are delivered may have been previously inserted into vasculature of a patient and may be dwelling within the vasculature when the tube 12 and/or the guidewire 13 is advanced into the catheter assembly. The catheter 73 is an example catheter illustrated in FIG. 1F.

In some embodiments, the tube 12 and/or the guidewire 13 may be disposed within a housing 14, which may be configured to protect the tube 12 from damage and/or contamination from a surrounding external environment. In some embodiments, the housing 14 may be rigid or semi-rigid. In some embodiments, the housing 14 may be made of one or more of stainless steel, aluminum, polycarbonate, metal, ceramic, plastic, and another suitable material. In some embodiments, the housing 14 may include a proximal end 16, a distal end 18, and a slot 20. In some embodiments, the slot 20 may extend parallel to a longitudinal axis of the housing 14.

In some embodiments, the instrument delivery device 10 may include an advancement element 22, which may extend through the slot 20 and may be configured to move linearly along the slot 20 between a retracted position illustrated, for example, in FIG. 1A, and an advanced position distal to the retracted position. In some embodiments, the retracted position may correspond to a fully retracted position with the advancement element 22 at a proximal end of the slot 20. In some embodiments, the clinician may pinch or grasp the advancement element 22 to move the advancement element 22 between the retracted position and the advanced position.

In some embodiments, the distal end 18 of the housing 14 may include a distal connector 24. In some embodiments, the distal connector 24 may include opposing lever arms 26a,26b. In some embodiments, distal ends of the opposing lever arms 26a,26b may be configured to move apart from each other in response to pressure applied to proximal ends of the opposing lever arms 26a,26b. In some embodiments, in response to removal of the pressure applied to the proximal ends of the opposing lever arms 26a,26b, the distal ends of the opposing lever arms 26a,26b may move closer to each other and clasp a portion of the catheter assembly, such as a needleless connector, another connector, or a proximal end of a catheter hub, for example. In some embodiments, the distal connector 24 may include a blunt cannula or male luer configured to insert into the portion of the catheter assembly.

In some embodiments, the distal connector 24 may include any suitable connector. For example, the distal connector 24 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 24 may include one or more bond pockets, which may each be configured to receive an extension tube, which may be part of the catheter assembly or extend between the distal connector 24 and the catheter assembly. In some embodiments, the distal connector 24 may be monolithically formed as a single unit with a body of the housing 14 that includes the slot 20.

In some embodiments, the tube 12 and/or the guidewire 13 may be replaced with any suitable probe or instrument. In some embodiments, the tube 12, the guidewire 13, or the suitable probe or instrument may include one or more sensors for patient or device monitoring and may include sensors measuring pressure, temperature, pH, blood chemistry, oxygen saturation, flow rate, or another physiological property.

In some embodiments, the guidewire 13 may include a first end 27 and a second end 28. In some embodiments, the first end 27 of the guidewire 13 may be secured within the housing 14. For example, the first end 27 of the guidewire 13 may be fixed to an inner surface of the housing 14. In some embodiments, in response to movement of the advancement element 22 distally a first distance along the slot 20, the second end 28 of the guidewire 13 may be configured to advance a second distance. In some embodiments, the second distance may be twice the first distance. In these embodiments, the advancement element 22 and the guidewire 13 may have a 1:2 advancement ratio such that for a particular distance the advancement element 22 is moved along the slot 20, the second end 28 of the guidewire 13 is moved twice the particular distance.

In some embodiments, the guidewire 13 may include any suitable shape. For example, the guidewire 13 may include a coil. In some embodiments, the second end 28 of the guidewire may be blunt and/or rounded to prevent damage to vasculature of a patient.

In some embodiments, the tube 12 may include a distal end 30 and a proximal end 32. In some embodiments, in response to movement of the advancement element 22 distally the first distance along the slot 20, the distal end 30 of the tube 12 may be configured to advance the first distance. In these embodiments, the advancement element 22 and the tube 12 may have a 1:1 advancement ratio such that for a particular distance the advancement element 22 is moved along the slot 20, the distal end 30 of the tube 12 is moved a distance equal to the particular distance.

In some embodiments, the proximal end 32 of the tube 12 may be coupled to the advancement element 22. In some embodiments, the instrument delivery device 10 may include an extension tube 34 coupled to the advancement element 22, and a blood collection pathway 36 may extend through the tube 12, the advancement element 22, and the extension tube 34.

In some embodiments, the instrument delivery device 10 may include a seal 38 disposed within the advancement element 22 and preventing fluid communication between the blood collection pathway 36 and a portion of a guidewire pathway through which the guidewire 13 moves. In some embodiments, the seal 38 may include an elastomeric septum. In some embodiments, the guidewire 13 may include the coil, which may be distal to the seal 38 or extend through the seal 38 when the advancement element 22 is in an initial or fully retracted position.

In some embodiments, in response to movement of the advancement element 22 distally the first distance along the slot 20, the second end 28 of the guidewire 13 and the distal end 30 of the tube 12 may move from inside the housing 14 to outside of the housing 14. In some embodiments, in response to the advancement element 22 being disposed at a proximal end of the slot 20, the second end 28 of the guidewire 13 and the distal end 30 of the tube 12 may be aligned with or proximal to a distal end 40 of the blunt cannula or male luer of the distal connector 24, which may protect the guidewire 13 and the tube 12 and prevent contamination thereof.

In some embodiments, the inner surface 42 of the housing 14 may include one or more grooves, which may be disposed between the proximal end 16 of the housing 14 and the distal end 18 of the housing 14. For example, the inner surface 42 may include a first groove 44 and/or a second groove 46. In some embodiments, the first groove 44 and/or the second groove 46 may be disposed within the housing 14 between the proximal end 16 and the distal end 18. In some embodiments, the tube 12 may be disposed within the first groove 44, which may provide guidance of the tube 12. In some embodiments, the guidewire 13 may be disposed within the first groove 44 and the second groove 46, which may provide guidance for the guidewire 13. In some embodiments, the first groove 44 and/or the second groove 46 may include a support wall 48, another support wall 50 opposite the support wall, and a bottom 52 extending between the support wall 48 and the other support wall 50. In some embodiments, the first groove 44 and/or the second groove 46 may be open opposite the bottom 52. In some embodiments, the first groove 44 and/or the second groove 46 may be linear and/or configured to guide the guidewire 13 as the guidewire 13 is advanced distally and/or retracted proximally.

In some embodiments, the guidewire 13 may be disposed in the first groove 44 and/or the second groove. In some embodiments, the tube 12 may be disposed within the first groove 44. In some embodiments, the first groove 44 and/or the second groove 46 may extend from the distal end 18 towards the proximal end 16 along all or a portion of a path on which the advancement element 22 travels. In some embodiments, the instrument delivery device 10 may include a support feature, which may be configured to contact the tube 12 to prevent the tube 12 and/or the guidewire 13 from buckling. Some example support features are further described in U.S. patent application Ser. No. 17/701,124, filed Mar. 22, 2022, which is hereby incorporated by reference in its entirety.

In some embodiments, the advancement element 22 may include an arc-shaped channel 54, which may be U-shaped. In some embodiments, the guidewire 13 may extend and move through the arc-shaped channel 54. In some embodiments, in response to movement of the advancement element 22 a first distance, the second end 28 of the guidewire 13 may be configured to advance distally a second distance that is more than twice the first distance. In these and other embodiments, the guidewire 13 may extend through multiple arc-shapes.

Referring now to FIG. 1F, the instrument delivery device 10 may be coupled to a catheter assembly 56. In some embodiments, the catheter assembly 56 may include an adapter 58, which may include a distal end 60 and a proximal end 62 aligned with the distal end 60 of the adapter 58. In some embodiments, the adapter 58 may include a side port 64 disposed between the distal end 60 of the adapter 58 and the proximal end 62 of the adapter 58. In some embodiments, the adapter 58 may include an adapter lumen 66, which may extend through the distal end 60 of the adapter 58, proximal end 62 of the adapter 58, and the side port 64.

In some embodiments, the catheter assembly 56 may include a catheter hub 68, which may include a distal end 70, a proximal end 72, a catheter hub lumen 74 extending through the distal end 70 of the catheter hub 68 and the proximal end 72 of the catheter hub 68. In some embodiments, the catheter 73 may extend from the distal end 70 and be secured within the catheter hub 68. In some embodiments, the catheter assembly 56 may be straight, and the instrument delivery device 10 may be coupled to and/or aligned with the proximal end 72 of the catheter hub 68. In other embodiments, the catheter hub 68 may include another side port 76 disposed in fluid communication with the catheter hub lumen 74. In some embodiments, septum 77 may be disposed within the catheter hub lumen 74 proximal to the other side port 76.

In some embodiments, the catheter assembly 56 may include an extension tube 78, which may include a distal end 80 and a proximal end 82. In some embodiments, the distal end 80 of the extension tube 78 may be coupled to the other side port 76. In some embodiments, the distal end 60 of the adapter 58 may be coupled to the proximal end 82 of the extension tube 78.

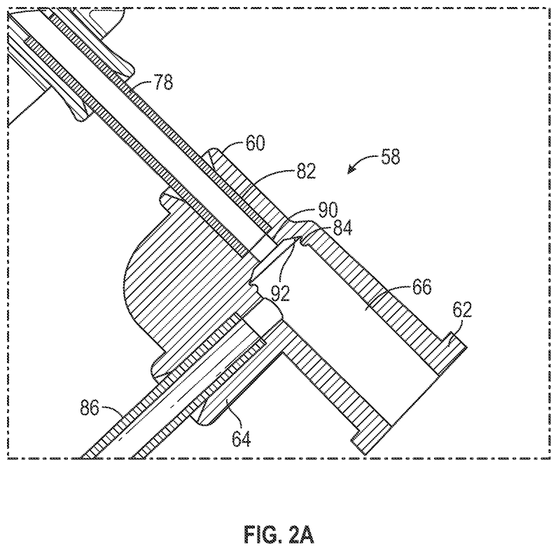

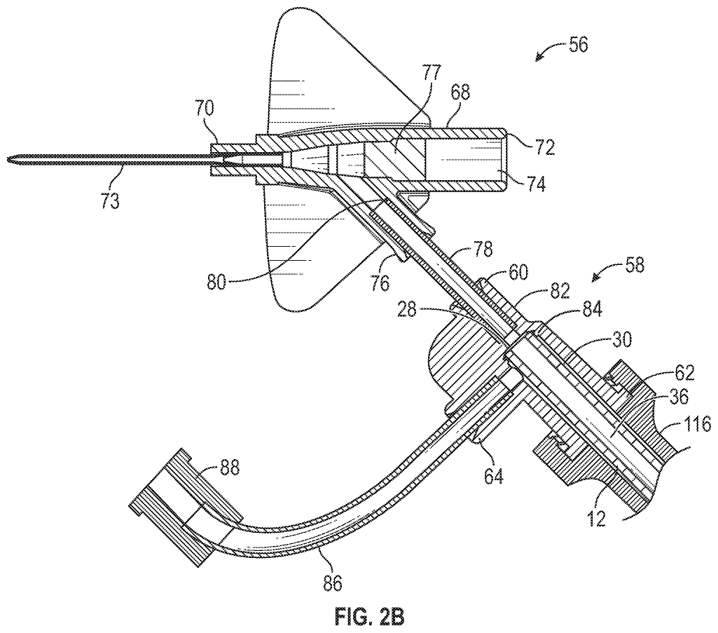

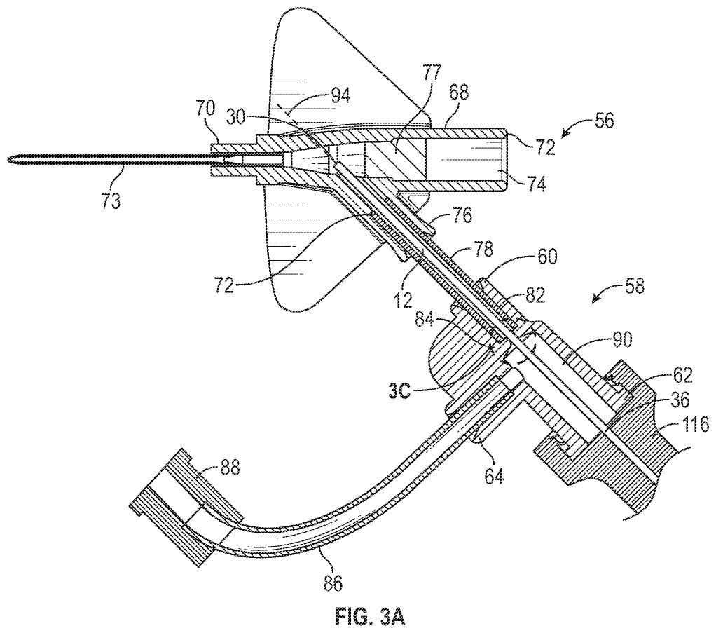

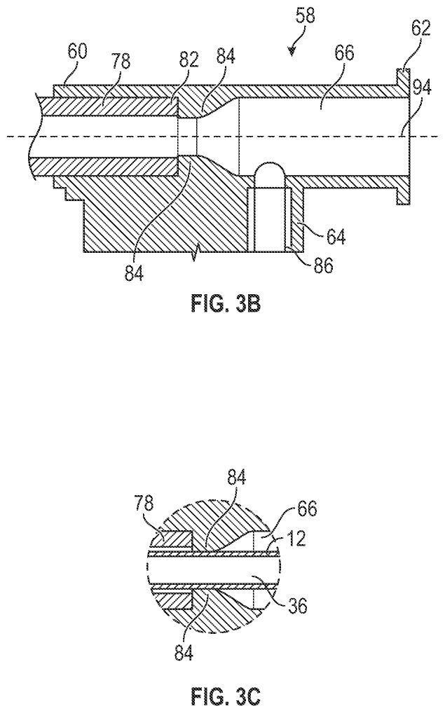

Referring now to FIGS. 2A-3C, in some embodiments, a portion of the adapter lumen 66 between the distal end 60 of the adapter 58 and the side port 64 may include an annular protrusion 84 configured to form a fluidic seal, which may decrease a likelihood of dilution or contamination of a blood collection pathway 36. In some embodiments, blood travelling through the blood collection pathway 36 may travel from vasculature of a patient proximally through the catheter 73, out the side port 64, through the extension tube 78, and into the tube 12 disposed within the adapter 58. In some embodiments, the fluidic seal may be formed around the tube 12 of the instrument delivery device 10 at the annular protrusion 84.

In some embodiments, the adapter 58 may include one or more other ports or openings in addition to the distal end 60, the proximal end 62, and the side port 64. In other embodiments, the adapter 58 may include a Y-connector or a T-connector, as illustrated in FIGS. 2A-3C. In some embodiments, the catheter assembly 56 may include another extension tube 86. In some embodiments, the side port 64 may be coupled to the other extension tube 86, which may be coupled to a connector 88. In some embodiments, the connector 88 may be configured to couple to an infusion device to infuse fluid distally through the catheter 73 into the vasculature of the patient.

As illustrated in FIG. 2A, in some embodiments, the portion of the adapter lumen 66 between the distal end 60 of the adapter 58 and the side port 64 may include a cylindrical uniform diameter portion 90 proximate the proximal end 82 of the extension tube 78 and proximate an outward taper 92. As illustrated in FIG. 2B, when the tube 12 and the advancement element 22 (see, for example, FIGS. 1A-1C), are in the advanced position, the tube 12 may terminate within the adapter 58, which may allow an increased inner diameter of the tube 12 and increase fluid flow rates therethrough. In these embodiments, the annular protrusion 84 may be proximal to the outward taper 92, which may facilitate the inner diameter of the tube 12 being large to increase fluid flow rates therethrough. Because termination of the tube 12 within the adapter 58 may also lead to dilution or contamination of the blood collection pathway 36 due to proximity to the side port 64, the fluidic seal may provide a light seal and separation between the blood collection pathway 36 and the side port 64.

As illustrated in FIGS. 3A-3C, in some embodiments, the annular protrusion 84 may be proximate the proximal end 82 of the extension tube 78. In some embodiments, an edge of the annular protrusion 84 proximate or contacting the proximal end 82 of the extension tube 78 may be disposed an equal or greater distance from a longitudinal axis 94 of the adapter 58 than an inner surface of the extension tube 78 proximate the annular protrusion 84, which may prevent blood from collecting or getting caught in between the annular protrusion 84 and the extension tube 78.

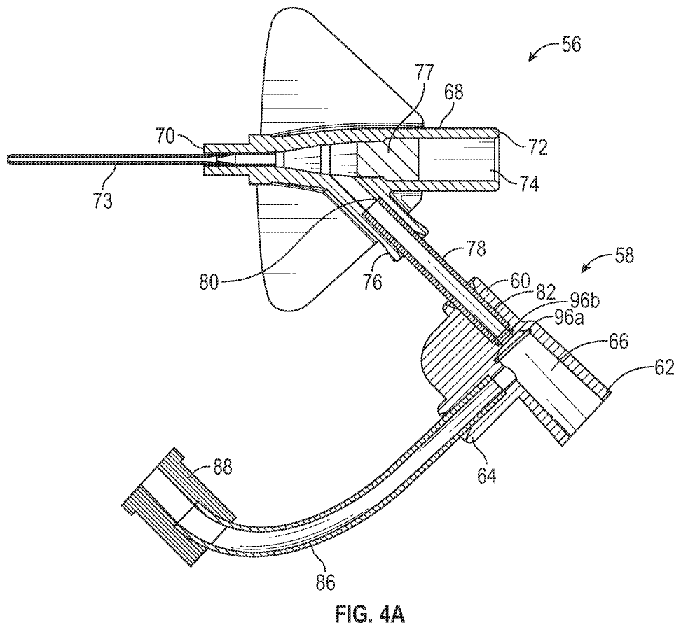

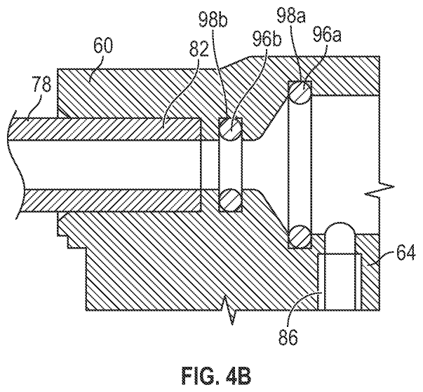

Referring now to FIGS. 4A-4B, in some embodiments, the catheter assembly 56 may include one or more annular elastomeric seals 96 disposed within the adapter lumen 66 between the proximal end 82 of the extension tube 78 and the side port 64. In some embodiments, the catheter assembly 56 may include an annular elastomeric seal 96a and/or another annular elastomeric seal 96b (which may be referred to collectively in the present disclosure as “elastomeric seals 96”), which may each be suited for providing the fluidic seal around different sizes of the tube 12.

In some embodiments, the annular elastomeric seals 96 may each be configured to form the fluidic seal around a particular size of tube 12 of the instrument delivery device 10. In some embodiments, the portion of the adapter lumen 66 between the distal end 60 of the adapter 58 and the side port 64 may include the cylindrical uniform diameter portion 90 proximate the outward taper 92, and the annular elastomeric seal 96a may be proximal to the outward taper 92. Additionally or alternatively, in some embodiments, the other annular elastomeric seal 96b may be disposed in the cylindrical uniform diameter portion 90 distal to the outward taper 92, and thereby configured to form the fluidic seal around a smaller particular tube 12 than the annular elastomeric seal 96a.

In some embodiments, an inner surface of the adapter 58 forming the adapter lumen 66 may include one or more annular grooves 98. In some embodiments, the annular elastomeric seal 96a and/or the other annular elastomeric seal 96b may each be seated within the annular grooves 98, which may allow the tube 12 to be larger and therefore blood flow therethrough increased. In some embodiments, the annular elastomeric seal 96a and/or the other annular elastomeric seal 96b may include an O-ring.

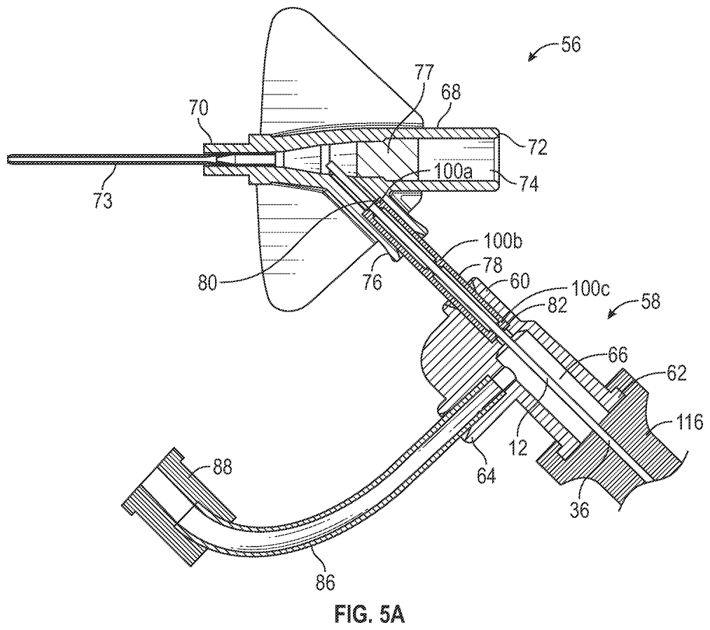

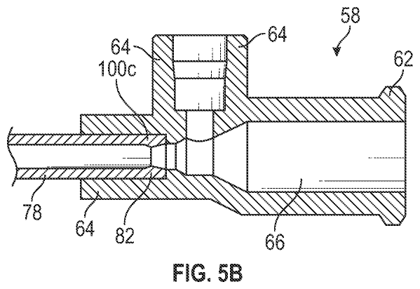

Referring now to FIGS. 5A-5B, in some embodiments, an inner surface of the extension tube 78 may include one or more annular protrusions 100 configured to form the fluidic seal around the tube 12 of the instrument delivery device 10. For example, the inner surface of the extension tube 78 may include one to three of the annular protrusions 100, which may allow the tube 12 to easily pass through without excessive friction. In some embodiments, the inner surface of the extension tube 78 may include more than three of the annular protrusions 100, which may facilitate strong separation and sealing between the blood collection pathway 36 and the side port 64.

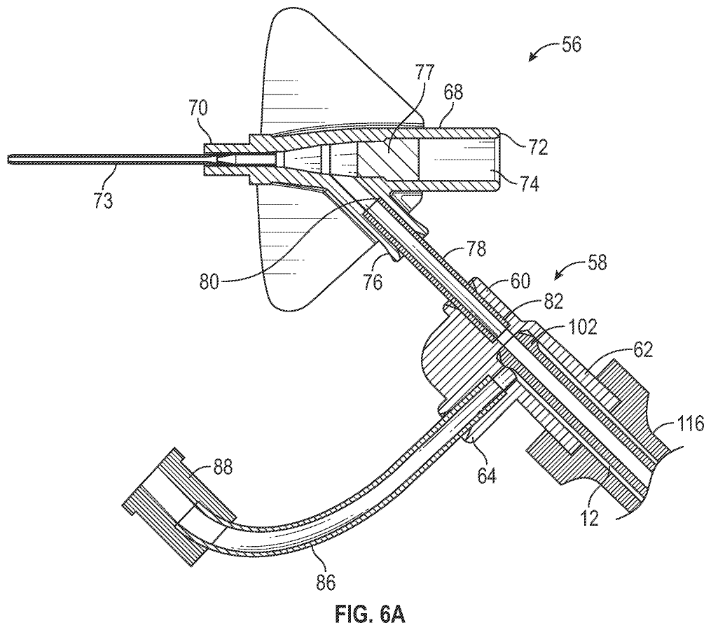

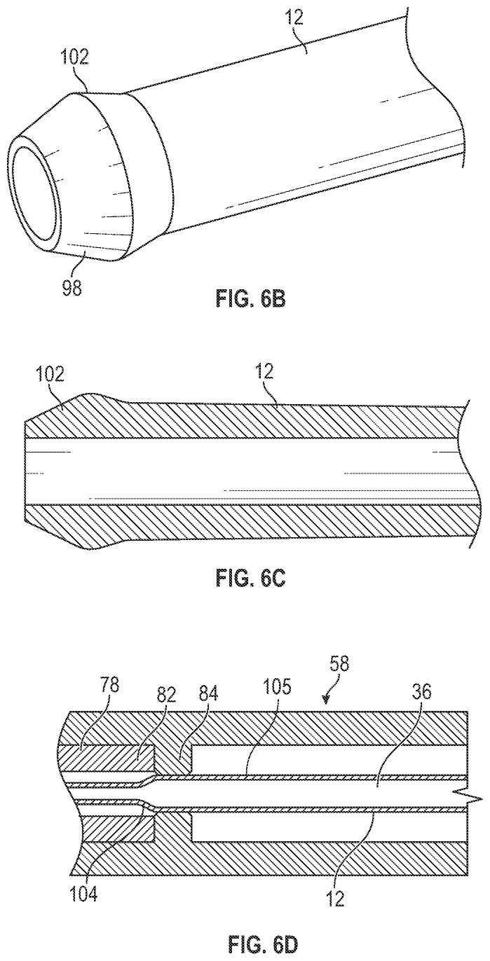

Referring now to FIGS. 6A-6C, in some embodiments, the distal end 30 of the tube 12 may include an annular protrusion 102, which may be configured to form the fluidic seal within the adapter 58 of the catheter assembly 56. In further detail, when the tube 12 and the advancement element 22 (see, for example, FIGS. 1A-1C), are in the advanced position, the tube 12 may terminate within the adapter 58 and the annular protrusion 102 form the fluidic seal with an inner surface of the adapter 58. In some embodiments, termination of the tube 12 within the adapter 58 may allow an increased inner diameter of the tube 12 and increase fluid flow rates therethrough. Moreover, the annular protrusion 102 may decrease a likelihood of dilution or contamination of the blood collection pathway 36, according to some embodiments.

Referring now to FIG. 6D, in some embodiments, the tube 12 may include a step or a taper 104 and an increased diameter portion 105 configured to form the fluidic seal within the adapter 58. In further detail, when the tube 12 and the advancement element 22 (see, for example, FIGS. 1A-1C), are in the advanced position, the increased diameter portion 105 may be disposed within the annular protrusion 84 within the adapter 58 and the annular protrusion 102 form the fluidic seal separating a fluid pathway of the side port 64 from the blood collection pathway 36. In these and other embodiments, the distal end 30 of the tube 12 may extend distal to the annular protrusion 102 when the tube 12 and the advancement element 22 are in the advanced position.

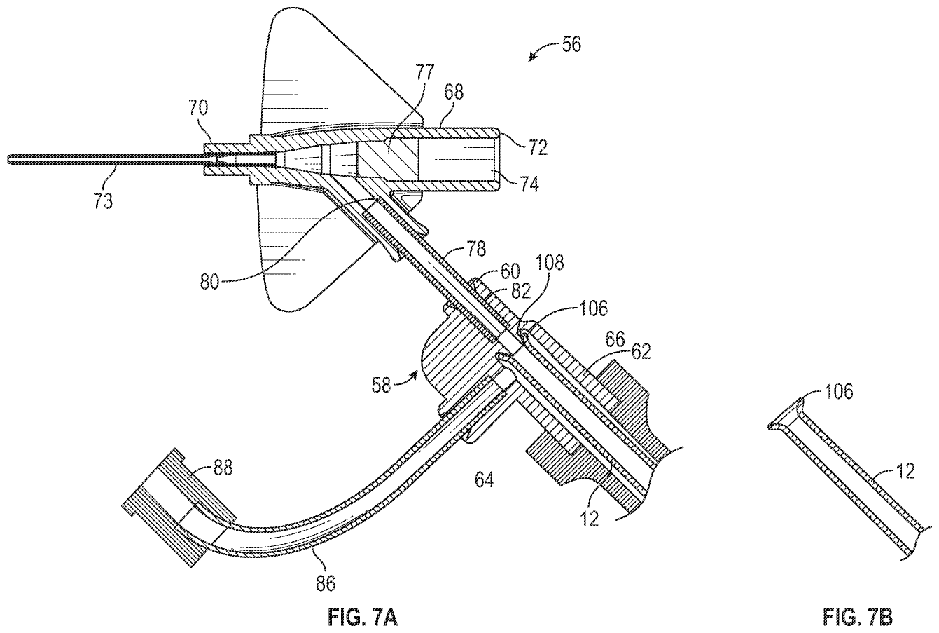

Referring now to FIGS. 7A-7B, in some embodiments, the distal end 30 of the tube 12 may include an outward flare 106. In some embodiments, an inner surface of the adapter 58 may include a proximally-extending protrusion 108, which may be annular. In some embodiments, the proximally-extending protrusion 108 may be configured to fit within the outward flare 106 when the tube 12 and the advancement element 22 (see, for example, FIGS. 1A-1C) are in the advanced position and form the fluidic seal.

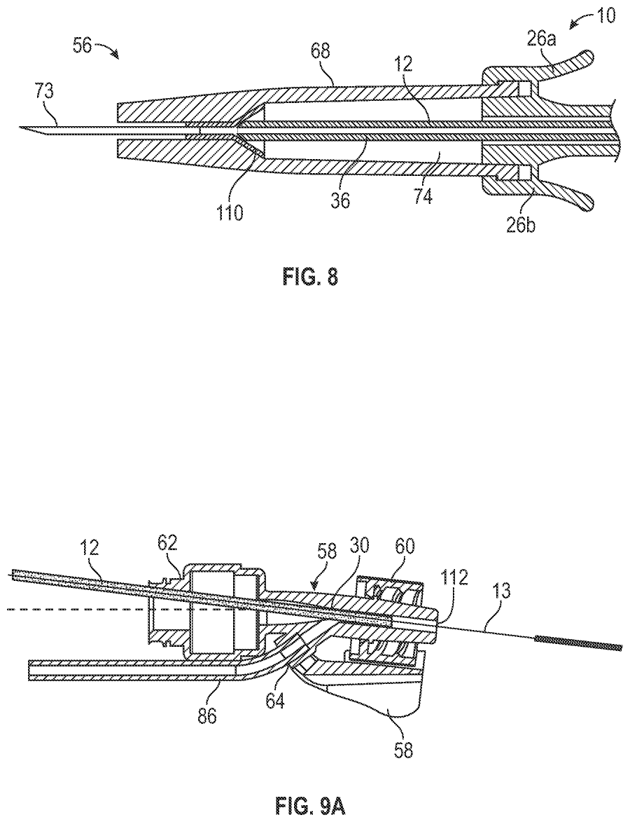

Referring now to FIG. 8, in some embodiments, when the tube 12 and the advancement element 22 (see, for example, FIGS. 1A-1C) are in the advanced position, the tube 12 may contact a wedge 110 that secures the catheter 73 and form the fluidic seal, which may prevent fluid from flowing through the wedge 110.

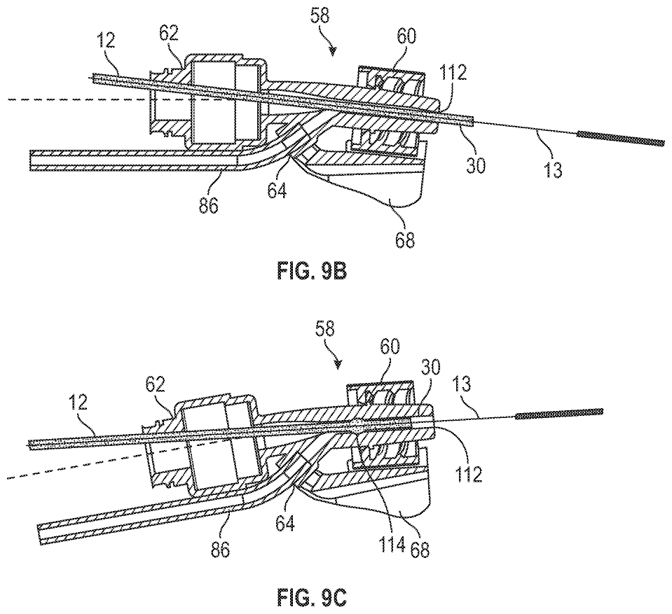

Referring now to FIG. 9A, in some embodiments, a method of blood collection may include advancing the tube 12 distally within the adapter 58. In some embodiments, the adapter 58 may include the side port 64. In some embodiments, in response to advancing the tube 12 distally within the adapter 58 of the catheter assembly 56, the fluidic seal may be formed around the tube 12 distal to the side port 64 within the adapter 58. In some embodiments, the distal end 60 of the adapter 58 may include an opening 112, and the fluidic seal may be formed proximal to the opening 112.

Referring now to FIG. 9B, in some embodiments, the fluidic seal may be at the opening 112, and in response to advancing the tube 12 distally within the adapter 58, an outer diameter of the tube 12 may contact the opening 112 to seal the opening 112 off and prevent liquid from flowing between the tube 12 and an inner surface of the adapter 58.

Referring now to FIG. 9C, in some embodiments, the tube 12 may include an annular protrusion 114, and in response to advancing the tube 12 distally within the adapter 58, the fluidic seal may be formed at the annular protrusion 114 of the tube 12.

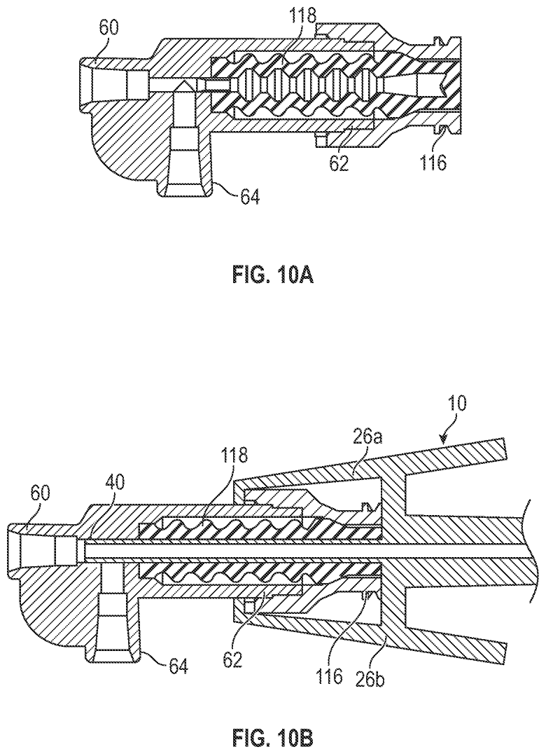

Referring now to FIGS. 10A-10B, in some embodiments, when the catheter assembly 56 is integrated, having the side port 64 and the extension tube 78, the catheter assembly 56 may include a needle-free connector 116 (also, illustrated, for example, in FIGS. 2B, 3A, 5A, 6A, and 7A) in fluid communication with the side port 64. In some embodiments, the side port 64 may be close to the needle-free connector, a septum 118 of the needle-free connector 116 extending into the adapter 58. In some embodiments, the blunt cannula or male luer of the instrument delivery device 10 may be lengthened to seal off the side port 64 and the distal end 40 may be disposed distal to the side port 64.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A catheter assembly, comprising:
an adapter comprising a distal end, a proximal end aligned with the distal end of the adapter, a side port disposed between the distal end of the adapter and the proximal end of the adapter, and an adapter lumen extending through the distal end of the adapter, proximal end of the adapter, and the side port, wherein a portion of the adapter lumen between the distal end of the adapter and the side port comprises an annular protrusion configured to form a fluidic seal around a tube of an instrument advancement device;
a catheter hub, comprising a distal end, a proximal end, a catheter hub lumen extending through the distal end of the catheter hub and the proximal end of the catheter hub, and another side port disposed in fluid communication with the catheter hub lumen; and
an extension tube, comprising a distal end and a proximal end, wherein the distal end of the extension tube is coupled to the other side port, wherein the distal end of the adapter is coupled to the proximal end of the extension tube.

2. The catheter assembly of claim 1, wherein the adapter comprises a T-connector or a Y-connector.

3. The catheter assembly of claim 1, further comprising another extension tube, wherein the side port is coupled to the other extension tube.

4. The catheter assembly of claim 1, wherein the portion of the adapter lumen between the distal end of the adapter and the side port comprises a cylindrical uniform diameter portion proximate the proximal end of the extension tube and proximate an outward taper, wherein the annular protrusion is proximal to the outward taper.

5. The catheter assembly of claim 1, wherein the annular protrusion is proximate the proximal end of the extension tube.

6. The catheter assembly of claim 5, wherein an edge of the annular protrusion proximate the proximal end of the extension tube is disposed an equal or greater distance from a longitudinal axis of the adapter than an inner surface of the extension tube proximate the annular protrusion.

7. A catheter assembly, comprising:
an adapter comprising a distal end, a proximal end aligned with the distal end of the adapter, a side port disposed between the distal end of the adapter and the proximal end of the adapter, and an adapter lumen extending through the distal end of the adapter, proximal end of the adapter, and the side port;
an annular elastomeric seal disposed within the adapter lumen between the proximal end of the extension tube and the other side port, wherein the annular elastomeric seal is configured to form a fluidic seal around a tube of an instrument advancement device;
a catheter hub, comprising a distal end, a proximal end, a catheter hub lumen extending through the distal end of the catheter hub and the proximal end of the catheter hub, and another side port disposed in fluid communication with the catheter hub lumen; and
an extension tube, comprising a distal end and a proximal end, wherein the distal end of the extension tube is coupled to the other side port, wherein the distal end of the adapter is coupled to the proximal end of the extension tube.

8. The catheter assembly of claim 7, wherein an inner surface of the adapter forming the adapter lumen comprises an annular groove, wherein the annular elastomeric seal is seated within the annular groove.

9. The catheter assembly of claim 8, wherein the annular elastomeric seal comprises an O-ring.

10. The catheter assembly of claim 7, wherein a portion of the adapter lumen between the distal end of the adapter and the side port comprises a cylindrical uniform diameter portion proximate an outward taper, wherein the annular elastomeric seal is proximal to the outward taper.

11. A catheter assembly, comprising:
an adapter comprising a distal end, a proximal end aligned with the distal end of the adapter, a side port disposed between the distal end of the adapter and the proximal end of the adapter, and an adapter lumen extending through the distal end of the adapter, proximal end of the adapter, and the side port;
a catheter hub, comprising a distal end, a proximal end, a catheter hub lumen extending through the distal end of the catheter hub and the proximal end of the catheter hub, and another side port disposed in fluid communication with the catheter hub lumen;
an extension tube, comprising a distal end and a proximal end, wherein the distal end of the extension tube is coupled to the other side port, wherein an inner surface of the extension tube comprises an annular protrusion configured to form a fluidic seal around a tube of an instrument advancement device, wherein the distal end of the adapter is coupled to the proximal end of the extension tube.

12. The catheter assembly of claim 11, wherein the inner surface of the extension tube comprises another annular protrusion.

13. An instrument advancement device, comprising:
a housing, comprising a proximal end, a distal end, a lumen disposed between the proximal end and a distal end, and a slot disposed between the proximal end and the distal end;
an advancement element extending through the slot and configured to move linearly along the slot between a retracted position and an advanced position;
a tube comprising a distal end and a proximal end, wherein when the advancement element is moved linearly along the slot from the retracted position to the advanced position, the second end of the instrument is advanced beyond the distal end of the housing, wherein the distal end of the tube comprises an annular protrusion or an outward flare, wherein the annular protrusion and the outward flare are configured to form a fluidic seal within an adapter of a catheter assembly.

14. An instrument advancement device, comprising:
a housing, comprising a proximal end, a distal end, a lumen disposed between the proximal end and a distal end, and a slot disposed between the proximal end and the distal end;
an advancement element extending through the slot and configured to move linearly along the slot between a retracted position and an advanced position; and
a tube comprising a first end and a second end, wherein when the advancement element is moved linearly along the slot from the retracted position to the advanced position, the second end of the instrument is advanced beyond the distal end of the housing, wherein the tube comprises a an increased diameter portion configured to form a fluidic seal within an adapter of a catheter assembly.

15. A method of blood collection, comprising:
advancing a tube distally within an adapter of a catheter assembly, wherein the adapter comprises a side port, wherein in response to advancing the tube distally within the adapter of the catheter assembly, a fluidic seal is formed around the tube distal to the side port within the adapter.

16. The method of claim 15, wherein a distal end of the adapter comprises an opening, wherein the fluidic seal is formed proximal to the opening.

17. The method of claim 16, wherein the tube comprises an annular protrusion, wherein in response to advancing the tube distally within the adapter of the catheter assembly, the fluidic seal is formed at the annular protrusion.

18. The method of claim 15, wherein the adapter comprises a distal end, a proximal end aligned with the distal end of the adapter, the side port disposed between the distal end of the adapter and the proximal end of the adapter, and an adapter lumen extending through the distal end of the adapter, the proximal end of the adapter, and the side port, wherein a portion of the adapter lumen between the distal end of the adapter and the side port comprises an annular protrusion configured to form the fluidic seal in response to advancing the tube distally within the adapter of the catheter assembly.

19. The method of claim 15, wherein the adapter comprises a distal end, a proximal end aligned with the distal end of the adapter, the side port disposed between the distal end of the adapter and the proximal end of the adapter, and an adapter lumen extending through the distal end of the adapter, the proximal end of the adapter, and the side port, wherein an elastomeric O-ring is disposed within adapter lumen between the proximal end of the extension tube and the side port, wherein the elastomeric O-ring forms the fluidic seal in response to advancing the tube distally within the adapter of the catheter assembly.

20. The method of claim 15, wherein the adapter comprises a distal end, a proximal end aligned with the distal end of the adapter, the side port disposed between the distal end of the adapter and the proximal end of the adapter, and an adapter lumen extending through the distal end of the adapter, the proximal end of the adapter, and the side port, wherein an extension tube extends from the distal end of the adapter to a catheter adapter, wherein an inner surface of the extension tube comprises an annular protrusion that forms the fluidic seal around the tube of an instrument advancement device in response to advancing the tube distally within the adapter of the catheter assembly.
